(19) United States
(12) Patent Application Publication
(10) Pub. No.: US 2024/0153614 A1
(43) Pub. Date: May 9, 2024

(54) METHOD AND SYSTEM FOR PROVIDING COGNITIVE BEHAVIORAL THERAPY FOR PAIN PATIENT

(71) Applicant: EVEREX, Seoul (KR)

(72) Inventors: Chi Hyun CHOI, Hanam-si (KR); Chan YOON, Seoul (KR)

(21) Appl. No.: 18/531,481

(22) Filed: Dec. 6, 2023

Related U.S. Application Data
(63) Continuation of application No. PCT/KR2023/003807, filed on Mar. 22, 2023.

(30) Foreign Application Priority Data
Nov. 3, 2022 (KR) 10-2022-0145283
Dec. 15, 2022 (KR) 10-2022-0176242

(51) Int. Cl. G16H 20/70; G16H 10/20; G16H 50/20

(52) U.S. Cl. CPC G16H 20/70; G16H 10/20; G16H 50/20

(57) ABSTRACT

Described herein is a method of providing cognitive behavioral therapy, in order to solve the above-mentioned problems, the method may include providing, to a user terminal, a plurality of survey data for diagnosing a state of a pain patient, receiving, through the user terminal, survey response data to the plurality of survey data, specifying, based on the survey response data, a user group to which the pain patient belongs among a plurality of user groups categorized according to the state of the pain patient, and determining, among a plurality of therapy protocols, an initial therapy protocol corresponding to the user group.

### CROSS REFERENCE TO RELATED APPLICATION

The application is a Continuation application of International Application PCT/KR2023/003807 filed on Mar. 22, 2023, which claims priority to Korean Patent Application No. 10-2022-0145283, filed on Nov. 3, 2022, and Korean Patent Application No. 10-2022-0176242, filed on Dec. 15, 2022, the entire contents of which is incorporated herein for all purposes by this reference.

### FIELD OF THE INVENTION

The present invention relates to a method and system for providing personalized cognitive behavioral therapy to a pain patient.

### DESCRIPTION OF THE RELATED ART

Cognitive behavioral therapy (CBT) is a psychological therapy method that works on the premise that a person's thoughts, feelings, and behaviors are closely interconnected, and in the course of therapy, patients themselves recognize their feelings, identify faulty thoughts, and change problematic behaviors.

In recent years, the cognitive behavioral therapy has been increasingly used to treat mental health issues such as depression, anxiety, insomnia, and pain, as the effects of cognitive behavioral therapy have been scientifically proven.

Meanwhile, as technology advances, electronic devices (e.g., smartphones, tablet PCs, etc.) have become more popular, and accordingly, there is an increasing dependency on the Internet for many aspects of daily life.

As such, with the development of various technologies, including the Internet, behavioral patterns that were previously highly dependent on offline activities have gradually shifted to online, and currently, the activities centered around the online world have been experiencing exponential growth.

In response to this trend, even offline-based fields of industry, such as the healthcare industry, are increasingly attempting to provide healthcare services online.

In particular, in recent years, various healthcare services have been provided online, and patients, i.e., users, have been able to have medical consultations with healthcare providers about their illnesses with just a few clicks on an internet-connected electronic device.

As an example of such technology, Korean Patent No. 10-2195512 discloses a technology related to a server and system that provides an online healthcare platform and provides information on healthcare service provision points to patients online.

### DISCLOSURE

### Technical Problem

The present invention relates to a method and system for providing cognitive behavioral therapy for a pain patient based on a digital device.

In particular, the present invention relates to a method and system for providing cognitive behavioral therapy that is capable of providing personalized cognitive behavioral therapy in consideration of patient's characteristics.

More specifically, the present disclosure relates to a method and system for providing cognitive behavioral therapy that is capable of providing personalized cognitive behavioral therapy that reflects a patient's pain duration and degree of cognitive distortion.

Further, the present invention relates to a method and system for providing cognitive behavioral therapy that is capable of providing updated cognitive behavioral therapy to a pain patient based on the pain patient's remission as the cognitive behavioral therapy progresses.

### Technical Solution

To solve the above-mentioned objects, there is provided a method for providing cognitive behavioral therapy according to the present invention, the method may include: providing, on a user terminal, a plurality of survey data for diagnosing a state of a pain patient; receiving, through the user terminal, survey response data for the plurality of survey data; detecting, based on the survey response data, state information of the pain patient related to pain duration and a degree of cognitive distortion of the pain patient; specifying a user group corresponding to the state information of the pain patient among a plurality of user groups categorized according to the pain duration and degree of cognitive distortion; determining an initial therapy protocol corresponding to the user group among a plurality of therapy protocols; and providing sequentially a plurality of specific therapy programs included in the initial therapy protocol to the user terminal, according to a therapy week set for each of the plurality of specific therapy programs.

The therapy protocol according to the present invention may be understood as a therapy process configured with a plurality of therapy programs, each matched to a different topic. Further, the specific therapy program may be configured to include at least one therapy module related to a specific topic matched to the specific therapy program. Here, the therapy module may be understood as a detailed category for cognitive behavioral therapy of the pain patient for a specific topic. According to an embodiment, a server may have a plurality of therapy programs stored that are each matched to a different topic, in which the plurality of therapy programs may be configured to include at least one therapy module related to the topic matched to each of the plurality of therapy programs, and in which each of the plurality of therapy protocols may be configured with at least one different therapy program or therapy module depending on characteristics of pain duration and degree of cognitive distortion of a user group matched to each of the plurality of therapy protocols.

In this case, the plurality of user groups may include: a first user group having acute pain and high cognitive distortion, in relation to the characteristics of pain duration and degree of cognitive distortion; a second user group having acute pain and low cognitive distortion, in relation to the characteristics of pain duration and degree of cognitive distortion; a third user group having chronic pain and high cognitive distortion, in relation to the characteristics of pain duration and degree of cognitive distortion; and a fourth user group having chronic pain and low cognitive distortion, in relation to the characteristics of pain duration and degree of cognitive distortion, in which a reference for distinguishing between acute pain and chronic pain may be determined by whether the pain duration exceeds a reference period, and in which a reference for distinguishing between high cognitive distortion and low cognitive distortion may be determined by whether a score collected based on a plurality of questions related to cognitive distortion exceeds a reference score.

According to an embodiment, the providing sequentially to the user terminal may include: providing sequentially, to the user terminal, specific therapy modules constituting the plurality of specific therapy programs, according to a therapy week set for each of the plurality of specific therapy programs; and collecting, from the user terminal, therapy response data from the specific therapy modules, and may further include: updating, using the therapy response data, the initial therapy protocol.

According to an embodiment, the updating of the initial therapy protocol may be configured to update the initial therapy protocol using initial therapy response data required by the therapy modules provided for each therapy week of a preset initial therapy period among a preset overall therapy period during which the plurality of specific therapy programs are scheduled to be provided to the user terminal, and in which the updating of the initial therapy protocol may be accomplished through changing at least one of remaining therapy programs allocated for a remaining therapy period of the overall therapy period excluding the initial therapy period and therapy modules constituting the remaining therapy programs.

According to an embodiment, the updating of the initial therapy protocol may include: analyzing, using the initial therapy response data, the state of the pain patient for each of a plurality of different analysis categories related to at least one of emotion, pain, insomnia, cognitive distortion, or stress; specifying, using an analysis result, a category of the plurality of analysis categories for which a problematic symptom of the pain patient satisfies a preset reference; and changing at least one of the remaining therapy programs allocated for the remaining therapy period or the therapy modules constituting the remaining therapy programs, to be related to the specified category.

According to an embodiment, as a result of updating the initial therapy protocol, at least some of example sentences provided from the therapy modules constituting the remaining therapy programs may be changed to be related to the specified category.

According to an embodiment, the updating of the initial therapy protocol may include: performing to judge, based on the therapy response data, whether to maintain the preset overall therapy period for which the plurality of specific therapy programs are scheduled to be provided to the user terminal; and performing, based on a judgment result, to update the preset overall therapy period, and in which the update of the preset overall therapy period may include shortening the overall therapy period to a period shorter than the overall therapy period or lengthening the overall therapy period to a period longer than the overall therapy period. According to an embodiment, each of the plurality of specific therapy programs may include a worksheet module configured to review at least one of a degree of pain, pain duration, a mental health state, or a physical health state of the pain patient, and in which in the providing sequentially to the user terminal, user response information for a worksheet module provided in a therapy week prior to a current therapy week of the plurality of specific therapy programs may be provided in priority before a therapy program corresponding to the current therapy week of the plurality of specific therapy programs is provided, in order to make the pain patient aware of a past state of the pain patient.

Further, there may be provided a system for providing cognitive behavioral therapy according to the present invention, the system may include: a communication unit configured to perform communication with a user terminal; a storage unit configured to store a plurality of therapy programs each matched to a different topic; and a control unit configured to provide a plurality of survey data for diagnosing a state of a pain patient on the user terminal, in which the control unit may be configured to: receive, through the communication unit, response data to the plurality of survey data from the user terminal; detect, based on the response data, state information of the pain patient related to the pain duration and degree of cognitive distortion of the pain patient; specify a user group corresponding to the state information of the pain patient among a plurality of user groups categorized according to the pain duration and degree of cognitive distortion; determine an initial therapy protocol corresponding to the user group among a plurality of therapy protocols; and provide a plurality of specific therapy programs included in the initial therapy protocol, sequentially to the user terminal, according to a therapy week set for each of the plurality of specific therapy programs.

Further, there may be provided a program executed by one or more processes on an electronic device and stored on a computer-readable recording medium, in which the program may include instructions for performing of: providing, on a user terminal, a plurality of survey data for diagnosing a state of a pain patient; receiving, through the user terminal, response data to the plurality of survey data; detecting, based on the response data, state information of the pain patient related to the pain duration and degree of cognitive distortion of the pain patient; specifying a user group corresponding to the state information of the pain patient among a plurality of user groups categorized according to the pain duration and degree of cognitive distortion; determining an initial therapy protocol corresponding to the user group among a plurality of therapy protocols; and providing a plurality of specific therapy programs included in the initial therapy protocol to the user terminal sequentially, according to a therapy week set for each of the plurality of specific therapy programs.

### Advantageous Effects

As described above, a method and system for providing cognitive behavioral therapy for a pain patient according to the present invention can receive, through a user terminal, a plurality of survey response data, and, based on the survey response data, detect state information of the pain patient related to pain duration and a degree of cognitive distortion of the pain patient. Further, based on the state information of the pain patient, a therapy protocol for personalized cognitive behavioral therapy can be provided to the pain patient. As a result, the method and system of cognitive behavioral therapy according to the present invention can provide the pain patient with a personalized cognitive behavioral therapy program for the pain patient in consideration of the pain duration and degree of cognitive distortion of the pain patient even if the pain patient has the same disease, rather than providing the pain patient with a uniform cognitive behavioral therapy based on the disease of the pain patient. Further, the pain patient can be provided with the personalized cognitive behavioral therapy that fits his/her state.

Further, the method and system for providing cognitive behavioral therapy for a pain patient according to the present invention can sequentially provide a plurality of specific therapy programs according to a therapy week. This allows the pain patient to be provided with cognitive behavioral therapy in a systematic way so that the pain patient can complete the cognitive behavioral therapy without dropping out.

Further, the method and system for providing cognitive behavioral therapy for a pain patient according to the present invention can update the therapy program to help the cognitive behavioral therapy of the pain patient based on therapy response data collected in the process of performing the cognitive behavioral therapy, so that the updated cognitive behavioral therapy can be provided in consideration of the pain patient's remission, rather than the initially determined method of cognitive behavioral therapy being provided continuously.

### BRIEF DESCRIPTION OF THE DRAWINGS

FIG. 1 is a conceptual view for describing a system for providing cognitive behavioral therapy according to the present invention.

FIG. 2 is a flowchart for describing a method of providing cognitive behavioral therapy according to the present invention.

FIG. 3, FIG. 4, FIG. 5A, FIG. 5B, FIG. 6A, and FIG. 6B are conceptual views for describing a method of providing an initial therapy protocol in consideration of a pain patient's pain duration and degree of cognitive distortion.

FIG. 7, FIG. 8A, FIG. 8B, FIG. 8C, and FIG. 8D are conceptual views for describing a method of updating the initial therapy protocol.

### DETAILED DESCRIPTION OF THE INVENTION

Hereinafter, exemplary embodiments disclosed in the present specification will be described in detail with reference to the accompanying drawings. The same or similar constituent elements are assigned with the same reference numerals regardless of reference numerals, and the repetitive description thereof will be omitted. The suffixes “module”, “unit”, “part”, and “portion” used to describe constituent elements in the following description are used together or interchangeably in order to facilitate the description, but the suffixes themselves do not have distinguishable meanings or functions. In addition, in the description of the exemplary embodiment disclosed in the present specification, the specific descriptions of publicly known related technologies will be omitted when it is determined that the specific descriptions may obscure the subject matter of the exemplary embodiment disclosed in the present specification. In addition, it should be interpreted that the accompanying drawings are provided only to allow those skilled in the art to easily understand the embodiments disclosed in the present specification, and the technical spirit disclosed in the present specification is not limited by the accompanying drawings, and includes all alterations, equivalents, and alternatives that are included in the spirit and the technical scope of the present disclosure.

The terms including ordinal numbers such as “first,” “second,” and the like may be used to describe various constituent elements, but the constituent elements are not limited by the terms. These terms are used only to distinguish one constituent element from another constituent element.

When one constituent element is described as being “coupled” or “connected” to another constituent element, it should be understood that one constituent element can be coupled or connected directly to another constituent element, and an intervening constituent element can also be present between the constituent elements. When one constituent element is described as being “coupled directly to” or “connected directly to” another constituent element, it should be understood that no intervening constituent element is present between the constituent elements.

Singular expressions include plural expressions unless clearly described as different meanings in the context.

In the present application, it should be understood that terms “including” and “having” are intended to designate the existence of characteristics, numbers, steps, operations, constituent elements, and components described in the specification or a combination thereof, and do not exclude a possibility of the existence or addition of one or more other characteristics, numbers, steps, operations, constituent elements, and components, or a combination thereof in advance.

The present invention is directed to providing a cognitive behavioral therapy protocol personalized for a pain patient, in consideration of pain duration and degree of cognitive distortion for pain of the pain patient, and more particularly, to a method and system for providing cognitive behavioral therapy centered on a pain patient caused by a musculoskeletal disease.

For convenience of description, the present invention is described with a focus on “musculoskeletal diseases,” but is not necessarily limited thereto. That is, in the present invention, the cognitive behavioral therapy may include both cognitive behavioral therapy for a pain patient due to a musculoskeletal disease, as well as cognitive behavioral therapy for a pain patient due to a variety of diseases (e.g., cancer, diabetes, hypertension, etc.).

In the present invention, the term “pain patient” is used interchangeably with “patient” or “user” to refer to a patient experiencing pain due to a disease.

Meanwhile, in case of the pain patient, each patient may have various pain duration and degree of cognitive distortion for pain, even if the patient has the same disease (or illness). Accordingly, in the present invention, a personalized cognitive behavioral therapy protocol may be provided to a pain patient, reflecting the pain duration and degree of cognitive distortion for the patient's pain.

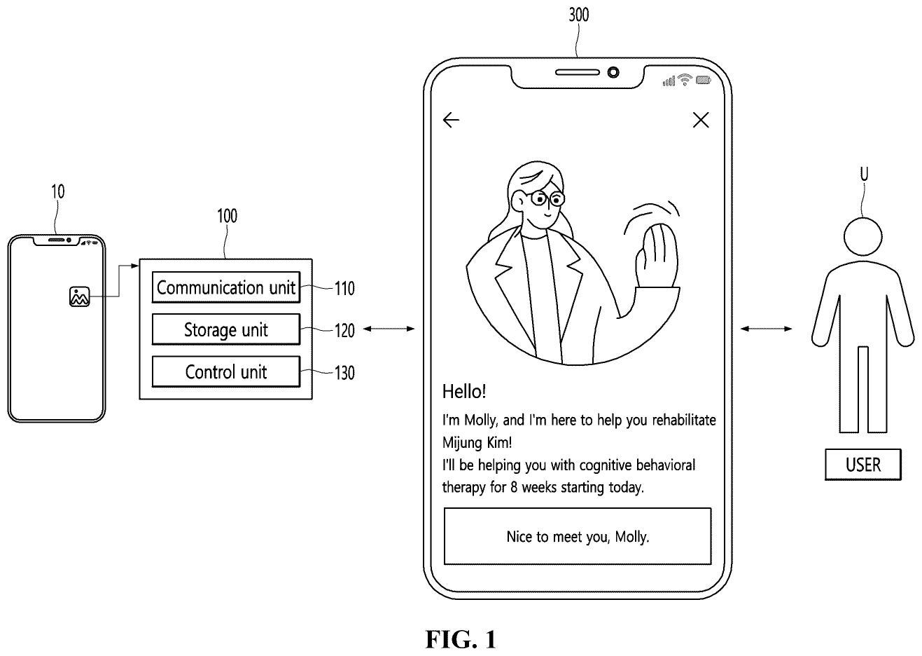

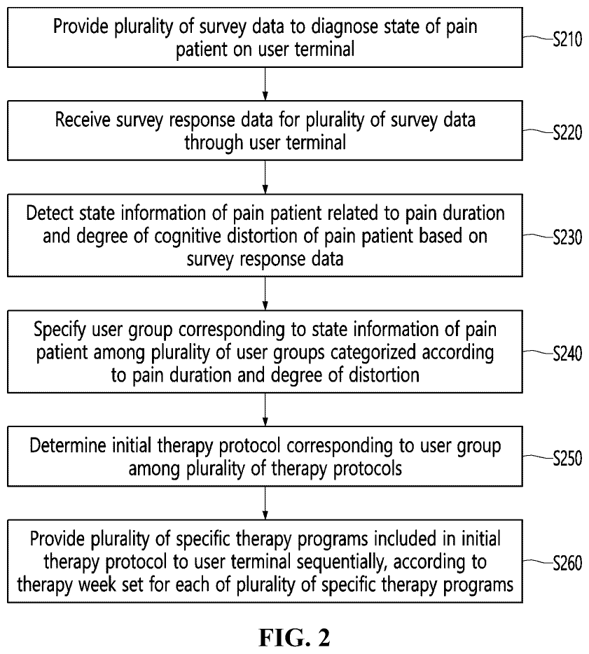

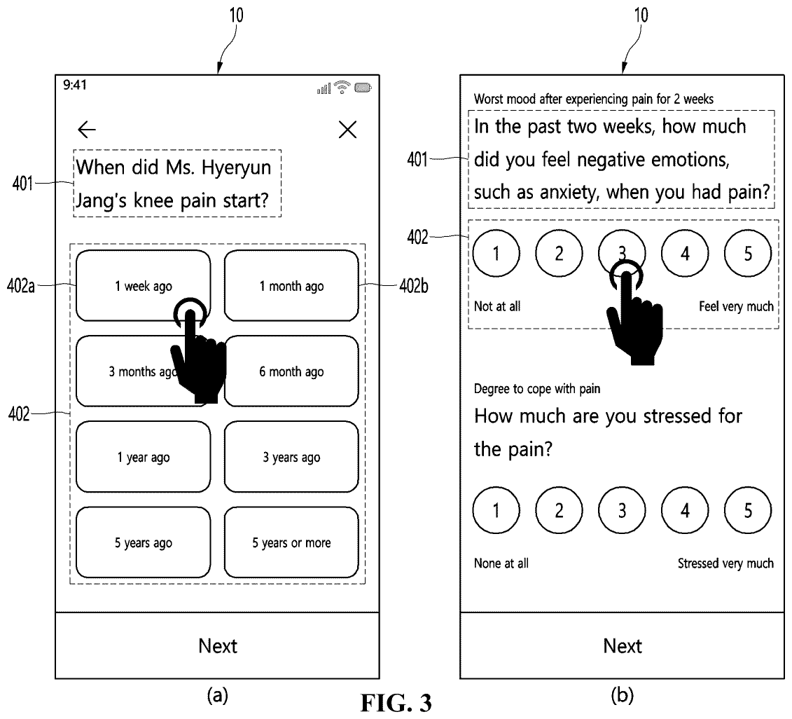

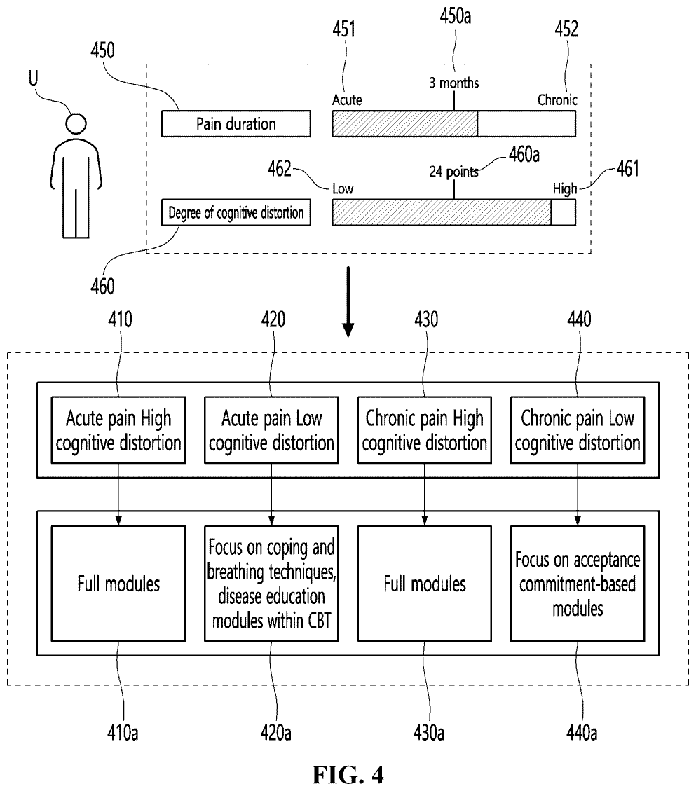

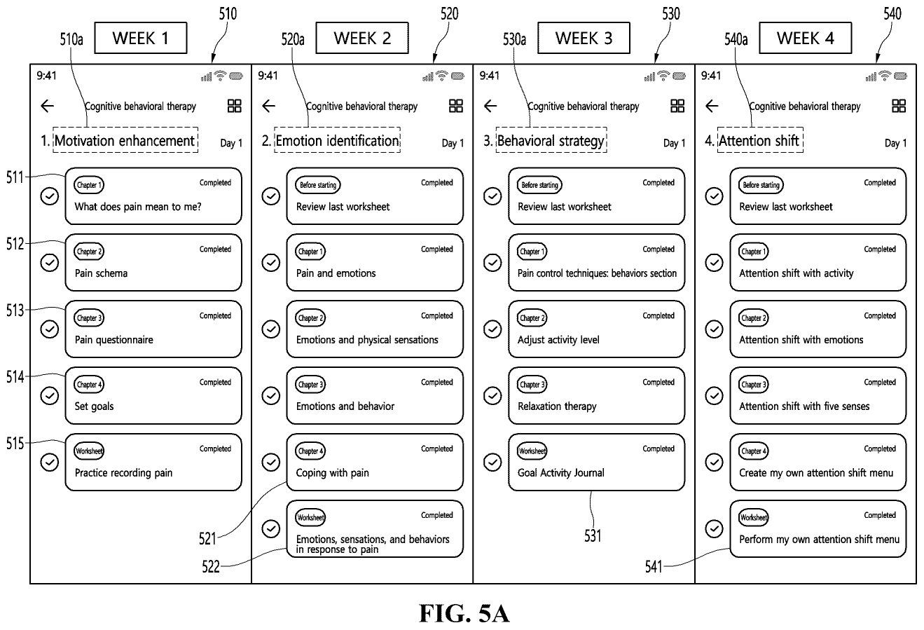

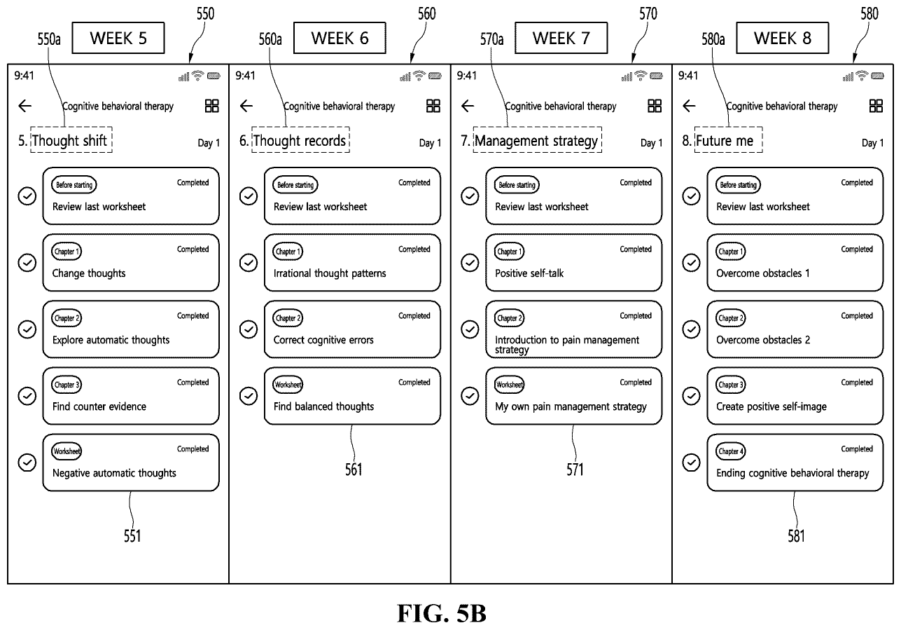

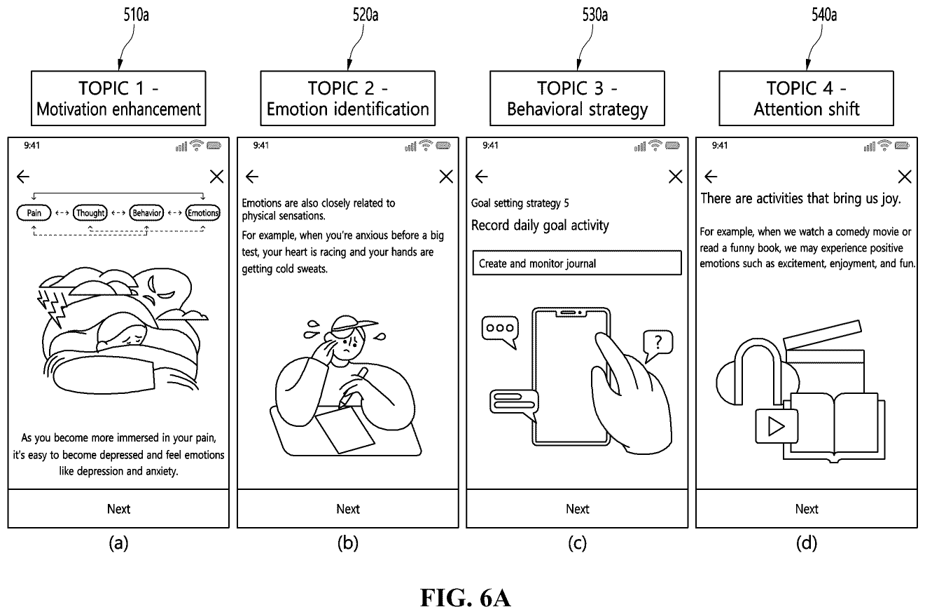

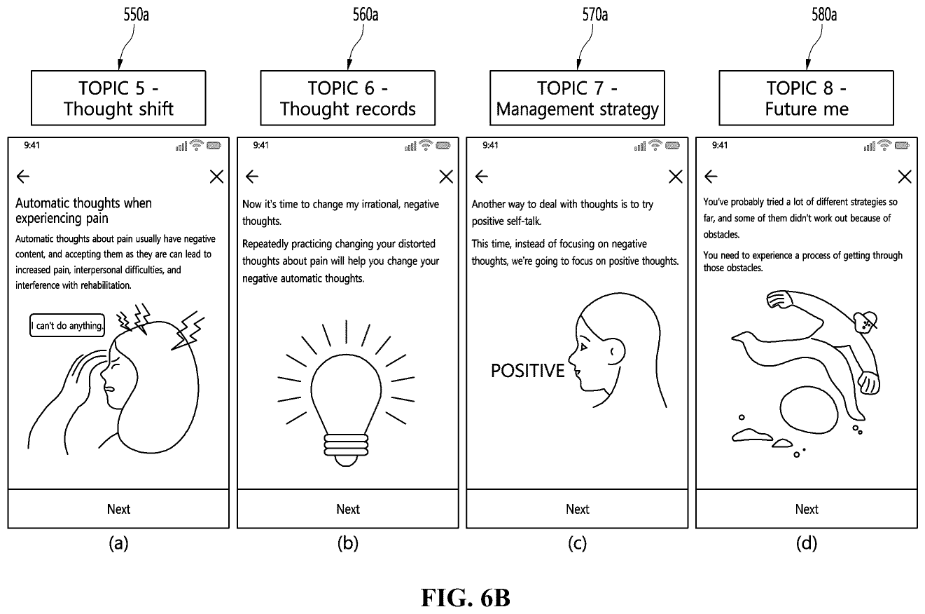

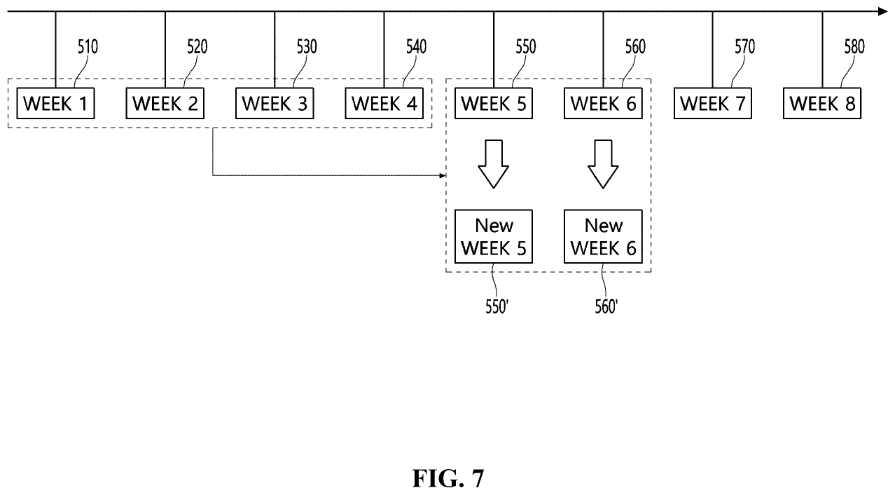

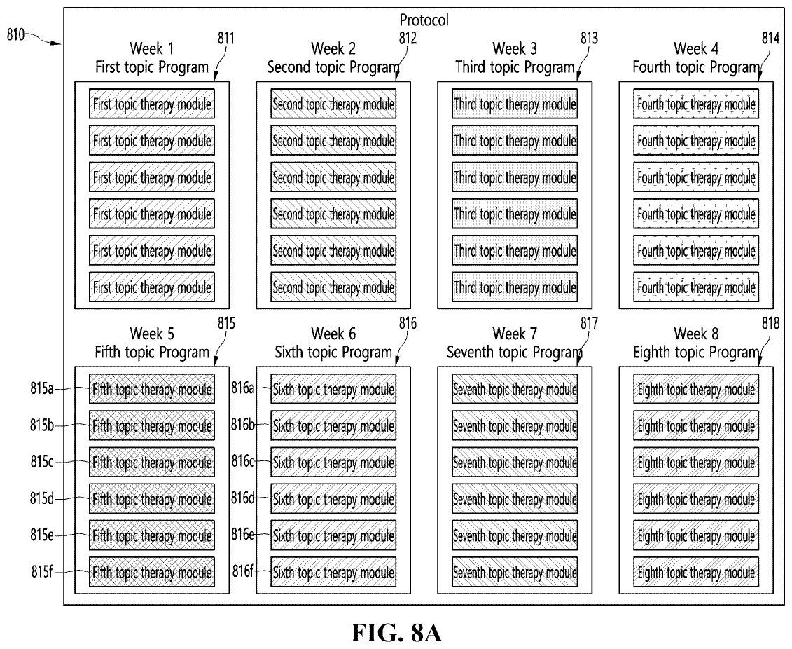

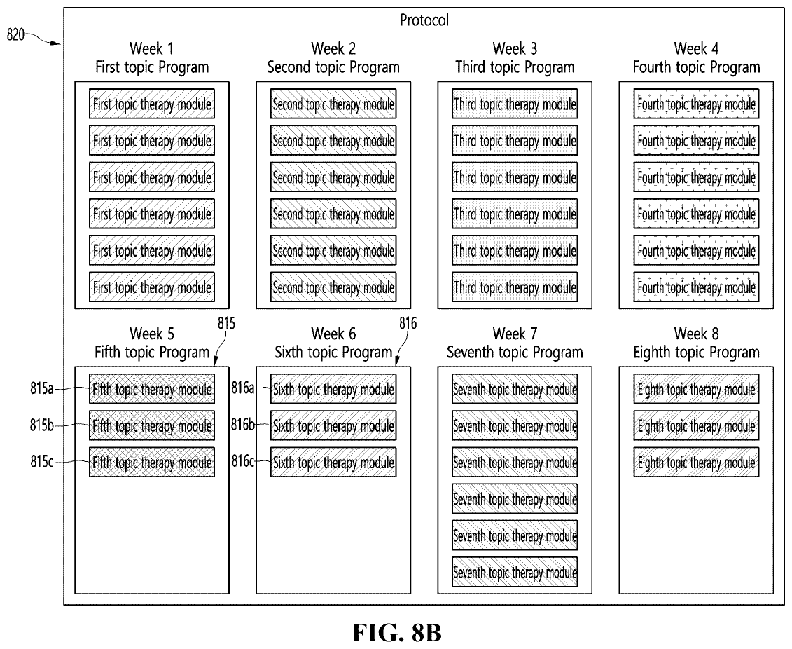

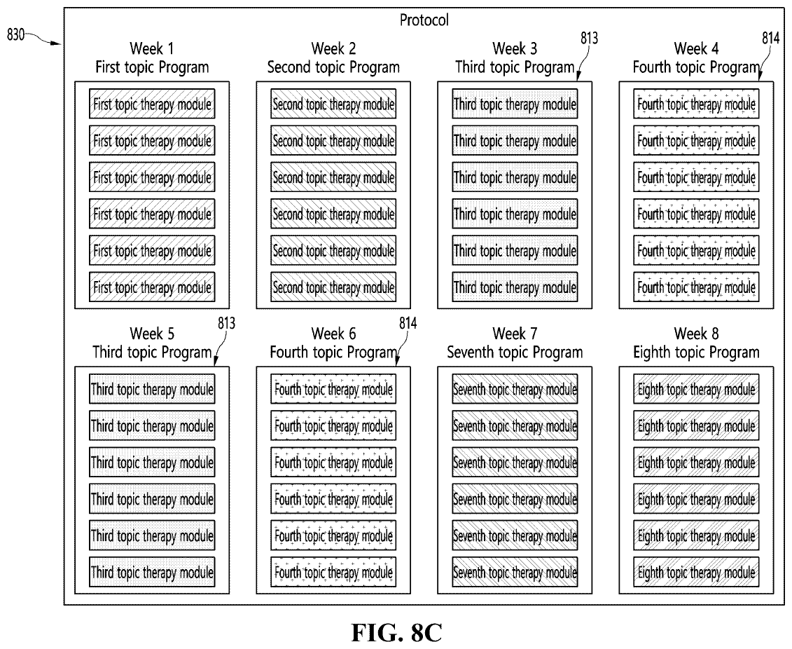

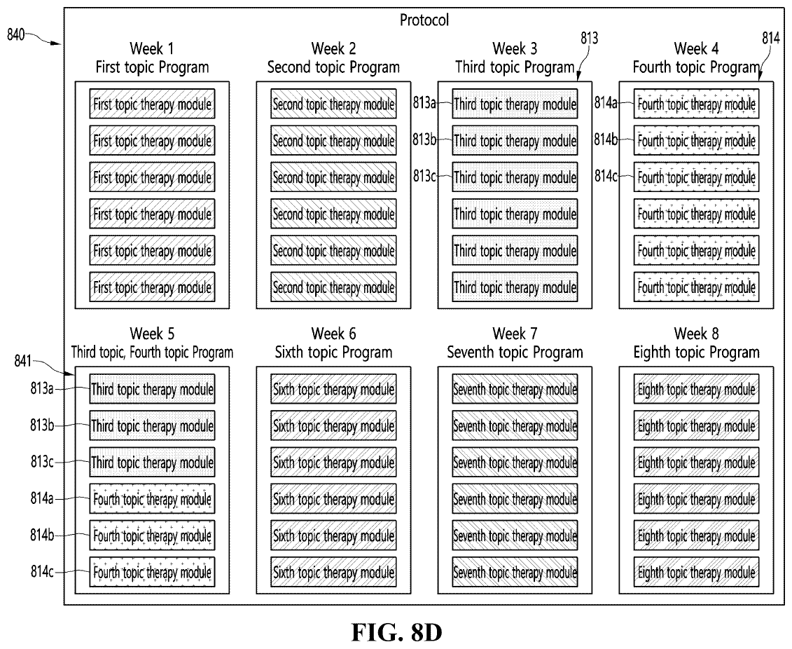

Hereinafter, with reference to the accompanying drawings, a method and system for providing a personalized cognitive behavioral therapy protocol for a pain patient will be described in detail. FIG. 1 is a conceptual view for describing a system for providing cognitive behavioral therapy according to the present invention. FIG. 2 is a flowchart for describing a method of providing cognitive behavioral therapy according to the present invention, FIG. 3, FIG. 4, FIG. 5A, FIG. 5B, FIG. 6A, and FIG. 6B are conceptual views for describing a method of providing an initial therapy protocol in consideration of a pain patient's pain duration and degree of cognitive distortion, and FIG. 7, FIG. 8A, FIG. 8B, FIG. 8C, and FIG. 8D are conceptual views for describing a method of updating the initial therapy protocol.

As illustrated in FIG. 1, a system for providing cognitive behavioral therapy for a pain patient according to the present invention (hereinafter referred to as a “cognitive behavioral therapy provision system” 100) may be implemented as an application or software.

According to a software implementation of a cognitive behavioral therapy provision system 100 according to the present invention, embodiments such as the procedures and functions described herein may be implemented as separate software therapy modules. Each of the software therapy modules may perform one or more of the functions and operations described herein.

As such, the software-implemented cognitive behavioral therapy provision system 100 may be downloaded to a user terminal 10 through a program that allows applications to be downloaded to the user terminal 10 (e.g., a play store), or may be implemented through an initial installation program on the user terminal 10. In this case, a communication unit 110, a storage unit 120, and a control unit 130 according to the present invention may be used as constituent elements of the user terminal 10.

In the present invention, the terminal 10 may also be referred to as a “mobile terminal” or an “electronic device”, and the terminal described herein may include a cell phone, a smart phone, a laptop computer, a digital broadcasting terminal, a personal digital assistant (PDA), a portable multimedia player (PMP), a navigation device, a slate PC, a tablet PC, an Ultrabook, a wearable device (e.g., a smartwatch, a smart glass, a head mounted display (HMD)), and the like.

Meanwhile, the cognitive behavioral therapy provision system 100 may exist inside a server built to perform a specific purpose (e.g., a function related to providing cognitive behavioral therapy) (hereinafter referred to as a server) separate from the user terminal 10, or may exist as a system separate from the server. When the cognitive behavioral therapy provision system 100 exists inside the server, a cognitive behavioral therapy service may be provided by at least one constituent element of the communication unit 110, storage unit 120, and control unit 130 located inside the server, or by a constituent module that performs a function similar to each of the constituent elements. In this case, the application may communicate with the server to provide services related to the cognitive behavioral therapy to the user terminal 10 on which the application is installed. Further, the cognitive behavioral therapy provision system 100 according to the present invention may provide a method of providing cognitive behavioral therapy according to the present invention to the user terminal 10 in conjunction with an external server. Meanwhile, the cognitive behavioral therapy provision system 100 according to the present invention may diagnose pain duration and degree of cognitive distortion for pain of a “patient” U using the user terminal 10, and provide a personalized cognitive behavioral therapy protocol in consideration thereof.

Further, the term “cognitive distortion” means a cognitive error that leads to a false assumption or misconception about pain, surrounding circumstances, events, etc. and the term “degree of cognitive distortion” may be understood as a term that expresses how much a patient is experiencing cognitive distortion. In the present invention, the term “degree of cognitive distortion” may be used interchangeably with “cognitive distortion state” and may be quantified as a “cognitive distortion score” or “cognitive distortion point”.

Meanwhile, the patient U may manage at least one of mental health and physical health states, such as diagnosing pain duration and degree of cognitive distortion for the patient's own pain, through an application or webpage provided by the cognitive behavioral therapy provision system 100 according to the present invention, and receiving counseling services according to the personalized cognitive behavioral therapy protocol through the user terminal 10.

In this case, the patient U may have a registered user account in the cognitive behavioral therapy provision system 100 according to the present invention. For convenience of description, an account of a user who is a patient is referred to herein as a “patient account” (or user account).

The “account” described above may be created through a page associated with the cognitive behavioral therapy provision system 100. In contrast, it is also possible that the “account” is created in at least one different system associated with the cognitive behavioral therapy provision system 100 according to the present invention.

Accordingly, in the present specification, all accounts based on the cognitive behavioral therapy provision system 100 according to the present invention will be referred to as an “account registered in the cognitive behavioral therapy provision system 100 according to the present invention” without distinguishing the system in which the account is issued.

Hereinafter, the cognitive behavioral therapy provision system 100 according to the present invention will be described according to an embodiment implemented as an application on the user terminal 10.

As illustrated in FIG. 1, the cognitive behavioral therapy provision system 100 for a pain patient according to the present invention may be configured to include at least one of the communication unit 110, the storage unit 120, or the control unit 130. In this case, the cognitive behavioral therapy provision system 100 according to the present invention is not limited to the constituent elements described above, and may further include constituent elements that serve the same function as the devices described herein.

The communication unit 110 may include one or more therapy modules to enable wireless or wired communication between the cognitive behavioral therapy provision system 100 and the user terminal 10, or between the cognitive behavioral therapy provision system 100 and an external server. In addition, the communication unit 110 may include one or more communication modules that connect the cognitive behavioral therapy provision system 100 to one or more networks.

Specifically, the communication unit 110 may collect, through the user terminal 10, survey response data for diagnosing a patient's pain duration and degree of cognitive distortion from at least one source. In addition, the communication unit 110 may provide a personalized cognitive behavioral therapy service according to the pain duration and degree of cognitive distortion of the pain patient through the user terminal 10.

The storage unit 120, which may also be referred to as a database (DB), is configured to store various information (or data) related to the cognitive behavioral therapy of the pain patient, such as the collected survey response data, a diagnosis result according to the survey response data, and a cognitive behavioral therapy protocol according to the diagnosis result.

Further, the cognitive behavioral therapy provision system 100 according to the present invention may use data stored in an external storage separately from the storage unit 120, and this external storage may also be referred to as a “database.”

Meanwhile, the control unit 130 may control an overall operation of the cognitive behavioral therapy provision system 100 according to the present invention.

The control unit 130 may control a page (or a service page 300) for providing a cognitive behavioral therapy service to be output through a display unit (or a touch screen) provided on the user terminal 10, as illustrated in FIG. 1. The page 300 may be output on the user terminal 10 through an application or webpage that is installed on the user terminal 10.

The page 300 is a page associated with the cognitive behavioral therapy provision system 100 according to the present invention, and is configured to be controlled by the cognitive behavioral therapy provision system 100 according to the present invention.

Further, when the page 300 is provided in the form of an application, the page 300 may be controlled by a central processing unit (CPU) of the user terminal 10 on which the application is installed. In this case, the CPU of the user terminal 10 may provide services related to cognitive behavioral therapy to the pain patient based on information provided by the cognitive behavioral therapy provision system 100 according to the present invention.

In the above, the constituent elements of the cognitive behavioral therapy provision system 100 according to the present invention have been described. Hereinafter, a method of providing a personalized cognitive behavioral therapy protocol to a pain patient based on the constituent elements described above will be described.

As illustrated in FIG. 2, a method of providing cognitive behavioral therapy according to the present invention may include a process of providing a plurality of survey data for diagnosing a state of a pain patient on the user terminal (S210).

The control unit 130 may control such that a page including a plurality of survey data is provided on the user terminal 10. In this case, the page including the plurality of survey data may be output through a touch screen (or a display) of the user terminal 10.

Here, the plurality of survey (or question, problem, statement, or test) data may include a survey for assessing a state on factors related to pain.

In the present invention, the factors related to pain may vary. For example, the factors related to pain may mean a variety of elements to assess a patient's state related to pain, such as degree of pain, pain duration, mental health, and physical health. However, it is obvious that the factors described above are just illustrative, and that the factors related to pain described in the present invention may mean any factors for evaluating the patient's state.

These factors related to pain may include validated survey data that are actually used in psychiatry to diagnose the patient's state. In addition, the control unit 130 may update the survey data frequently or periodically by downloading additional surveys for diagnosing the patient's state through an external server or website or the like. Hereinafter, with reference to FIG. 3, a user interface through which the survey data is provided is described. As illustrated in FIG. 3, a page including a survey area 401 for displaying at least one survey of the plurality of surveys and a response area 402 for receiving a patient's response to the displayed survey may be output on the user terminal 10. In this case, the response area 420 may include response graphic objects 402a and 402b corresponding to each of a plurality of different responses to the surveys displayed in the survey area 401.

Accordingly, in response to any one of the displayed surveys, the pain patient may select a candidate response that is believed to be the most appropriate (or appropriate, or accurate) for the user's state among candidate responses output in the response area 402 as the survey response data.

Meanwhile, in the present invention, a process of receiving the survey response data for the plurality of survey data may be performed via the user terminal (S220, see FIG. 2).

The control unit 130 may receive the survey response data from the user terminal 10 based on a user selection (or user input) for the response area 402 of the user terminal 10.

For example, as illustrated in FIG. 3A, based on one of the plurality of response graphic objects 402a and 402b corresponding to a first survey of the pain patient's pain duration (e.g., “When did Ms. Hyeryun Jang's knee pain start?”) being selected, a response (e.g., “1 week ago”) corresponding to the selected response graphic object 402a may be received as survey response data for the first survey. Meanwhile, the first survey may include a plurality of surveys. In this case, the user terminal 10 may be provided with a plurality of surveys necessary to detect state information on the patient's pain duration. Further, a response to the plurality of surveys may be received from the user terminal as the survey response data.

In another example, as illustrated in FIG. 3B, based on any one of the plurality of response graphic objects corresponding to a second survey related to a patient's degree of cognitive distortion being selected, a response (e.g., “3”) corresponding to the selected response graphic object may be received as survey response data for the second survey.

Meanwhile, the second survey may include a plurality of surveys. In this case, the user terminal 10 may be provided with a plurality of surveys necessary to detect state information on the pain patient's degree of cognitive distortion. Further, a response to the plurality of surveys may be received from the user terminal as the survey response data. An example of the second survey may include a survey related to at least one of pain catastrophizing scale or fear (or risk) avoidance beliefs, such as: i) I am always worried that the pain will not stop, ii) I feel like it is unbearable, iii) The pain is so painful that I think I will never get better, iv) My pain is caused by physical activity, v) Physical activity makes my pain worse, vi) My pain is caused by work or an accident at work, etc. Further, unlike the above, the plurality of survey data to diagnose the pain patient's state may further include a survey to judge depression, insomnia, etc.

Meanwhile, in the present invention, based on the survey response data, a process of detecting state information on the pain patient related to the pain duration and degree of cognitive distortion of the pain patient may be performed (S230, see FIG. 2).

As illustrated in FIG. 4, the control unit 130 may compare pain duration 450 extracted (obtained or detected) from the survey response data of the pain patient to a first reference (e.g., “3 months”, 450a) in order to detect the state information on the pain patient.

Here, the first reference 450a may be understood as a predefined reference period (e.g., “3 months”) for categorizing (or distinguishing) the pain duration (or the nature of the pain based on the pain duration) into one of “a first type of pain duration (e.g., acute pain, 451)” or “a second type of pain duration (e.g., chronic pain, 452)”.

When the pain duration 450 of the pain patient is shorter than the predefined reference period 450a (i.e., equal to or less than the predefined reference period) as a result of the comparison, the control unit 130 may categorize (distinguish, specify, or determine) the pain duration of the pain patient as a “first type of pain duration (e.g., acute pain, 451)”. In contrast, when the pain duration 450 of the pain patient is longer than the predefined reference period 450a (i.e., exceeds the predefined reference period), the control unit 130 may categorize the pain duration of the pain patient as a “second type of pain duration” (e.g., chronic pain, 452).

The control unit 130 may categorize the pain duration of the pain patient into one of acute pain or chronic pain, depending on whether the pain duration 450 exceeds the reference period 450a.

Further, the control unit 130 may compare a degree of cognitive distortion 460 extracted (obtained or detected) from the survey response data of the pain patient to a second reference 460a.

Here, the degree of cognitive distortion of the pain patient may be related to one of the pain catastrophizing scale or fear (or risk) avoidance beliefs, as described above. Here, the second reference 460a may be understood as a predefined reference score (e.g., “24 points”) for categorizing (or distinguishing) the degree of cognitive distortion of the pain patient into one of a “first type of degree of cognitive distortion (e.g., high cognitive distortion, 461)” and a “second type of degree of cognitive distortion (e.g., low cognitive distortion, 462).

The control unit 130 may calculate (derive or compute) a cognitive distortion score of the pain patient based on the survey response data to compare the degree of cognitive distortion 460 of the pain patient to the predefined reference score 460a.

A plurality of selection items on a survey to judge the degree of cognitive distortion (e.g., I am always worried that the pain will not stop) may each be matched with a different score (e.g., not at all: 0 point, a little: 1 point, usually: 2 points, a lot: 3 points, always: 4 points). The response data may be a selection signal for one of the plurality of selection items, and the control unit 130 may calculate (derive or compute) a cognitive distortion score of the pain patient using a score matched to the selection item according to the received selection signal.

Meanwhile, the control unit 130 may calculate a cognitive distortion score of the pain patient from the survey response data using various methods. For example, the control unit 130 may calculate a cognitive distortion score of the pain patient using a sum or average of the survey response data corresponding to the plurality of survey data. In this case, the control unit 130 may allocate (assign) different weights for each survey response data to calculate the cognitive distortion score of the pain patient.

The control unit 130 may compare the calculated cognitive distortion score to the predefined reference score 460a.

When the cognitive distortion score of the pain patient is higher than the predefined reference score 460a (i.e., above the predefined score) as a result of the comparison, the control unit 130 may categorize (distinguish, specify, or determine) the degree of cognitive distortion of the pain patient as the “first type of degree of cognitive distortion (high cognitive distortion, 461)”. In contrast, when the cognitive distortion score of the pain patient is lower than the predefined reference score 460a (i.e., equal to or less than the predefined cognitive distortion score), the control unit 130 may categorize the degree of cognitive distortion of the pain patient as the “second type of degree of cognitive distortion (low cognitive distortion, 462)”.

That is, the control unit 130 may determine the degree of cognitive distortion of the pain patient as one of high cognitive distortion or low cognitive distortion, depending on whether the cognitive distortion score exceeds the reference score.

Meanwhile, in the present invention, a process of specifying a user group corresponding to the state information on the pain patient among a plurality of user groups categorized according to pain duration and degree of cognitive distortion may be performed (S240, see FIG. 2).

As illustrated in FIG. 4, in the present invention, a plurality of user groups 410, 420, 430, and 440 may be categorized based on the pain duration 450 and degree of cognitive distortion 460. For convenience of description, in the present invention, an example is provided in which there are “four” different user groups based on the pain duration 450 and degree of cognitive distortion 460. Each of the user groups in the present invention may be referred to as a first user group 410, a second user group 420, a third user group 430, and a fourth user group 440. However, in the present invention, it is obvious that condition items and the number of conditions to categorize the user group may be set variously.

Meanwhile, in each of the plurality of user groups 410, 420, 430, and 440, categorization reference information that categorizes the plurality of user groups may exist to be matched. This categorization reference information may be predefined and exist on the storage unit 120 or on an external server.

Specifically, in the first user group 410, a first type of pain duration (e.g., acute pain, 451) and a first type of degree of cognitive distortion (e.g., high cognitive distortion, 461) may exist to be matched as the categorization reference information.

In the second user group 420, the first type of pain duration (e.g., acute pain, 451) and a second type of degree of cognitive distortion (e.g., low cognitive distortion, 462) may exist to be matched as the categorization reference information.

In the third user group 430, a second type of pain duration (e.g., chronic pain, 452) and the first type of degree of cognitive distortion (e.g., high cognitive distortion, 461) may exist to be matched as the categorization reference information.

Further, in the fourth user group 440, the second type of pain duration (e.g., chronic pain, 452) and the second type of degree of cognitive distortion (e.g., low cognitive distortion, 462) may exist to be matched as the categorization reference information.

The control unit 130 may determine, among the plurality of user groups 410, 420, 430, and 440, a specific user group in which the categorization reference information corresponding to the state information of the pain patient is matched.

Specifically, when the state information of a first pain patient (e.g., “patient Cheolsoo Kim”) includes the first type of pain duration (e.g., acute pain, 451) and the first type of degree of cognitive distortion (e.g., high cognitive distortion, 461), the control unit 130 may specify (or categorize) the first pain patient as a pain patient belonging to the first user group 410 among the plurality of user groups.

Further, when the state information detected from the survey response data of a second pain patient (e.g., “patient Youngsuk Lee”) includes the first type of pain duration (e.g., acute pain, 451) and the second type of degree of cognitive distortion (e.g., low cognitive distortion, 462), the control unit 130 may specify (or categorize) the second pain patient as a pain patient belonging to the second user group 420 among the plurality of user groups.

Further, when the state information of a third pain patient (e.g., “patient Mincheol Choi”) includes the second type of pain duration (e.g., chronic pain, 452) and the first type of degree of cognitive distortion (e.g., high cognitive distortion, 461), the control unit 130 may specify (or categorize) the third pain patient as a pain patient belonging to the third user group 430 among the plurality of user groups.

Further, when the state information of a fourth pain patient (e.g., “patient Minji Bang”) includes the second type of pain duration (e.g., chronic pain, 452) and the second type of degree of cognitive distortion (e.g., low cognitive distortion, 462), the control unit 130 may specify (or categorize) the fourth pain patient as a pain patient belonging to the fourth user group 440 among the plurality of user groups.

Meanwhile, in the present invention, a process of determining an initial therapy protocol corresponding to the user group among the plurality of therapy protocols may be performed (S250, see FIG. 2).

As illustrated in FIGS. 5A and 5B, the therapy protocol according to the present disclosure may be configured with a plurality of therapy programs 510 to 580, each matched with different topics 510a to 580a. That is, each therapy program means a program for performing therapy according to a topic matched to each therapy program.

Here, “topics 510a to 580a” correspond to the plurality of programs 510 to 580 for cognitive behavioral therapy of the pain patient, respectively. For example, as illustrated in FIGS. 6A and 6B, i) motivation enhancement (hereinafter, a first topic 510a) may correspond to a first program 510, ii) emotion identification (hereinafter, a second topic 520a) may correspond to a second program 520, iii) behavioral strategy (hereinafter, a third topic 530a) may correspond to a third program 530, iv) attention shift (hereinafter, a fourth topic 540a) may correspond to a fourth program 540, v) thought shift (hereinafter, a fifth topic 550a) may correspond to a fifth program 550, vi) thought record (hereinafter, a sixth topic 560a) may correspond to a sixth program 560, vii) management strategy (hereinafter, a seventh topic 570a) may correspond to a seventh program 570, and viii) future me (hereinafter, an eighth topic 580a) may correspond to an eighth program 580.

The first topic (motivation enhancement) is directed to understanding cognitive behavioral therapy and schema for pain and increasing therapy motivation by establishing the pain patient's own goals, and may be configured with therapy modules related thereto.

The second topic (emotion identification) is directed to identifying pain, emotions, and the pain patient's own coping styles, and may be configured with therapy modules to identify the pain patient's negative emotions related to pain, physical reactions, and behaviors, and to identify the pain patient's own coping styles.

The third topic (behavioral strategy) is therapy for pain patients to set new coping behavior ways by setting activity goals and to cope with pain using breathing and progressive relaxation techniques, which may be configured with therapy modules therefor.

The fourth topic (attention shift) is a therapy for coping with pain using an attention shifting method that uses activities, emotions, and the five senses, and may be configured with therapy modules therefor.

The fifth topic (thought shift) is directed to exploring negative automatic thoughts related to pain, identifying feelings and behaviors based on automatic thoughts, and engaging in a process of finding evidence to counter the automatic thoughts, which may be configured with therapy modules therefor.

The sixth topic (thought record) is a process of exploring the pain patient's own irrational thought patterns and finding cognitive flexibility, which may be configured with therapy modules therefor.

The seventh topic (management strategy) is directed to performing positive self-talk and practicing pain management strategy, which may be configured with therapy modules to use positive self-talk to cope with pain and to understand the “stop-think-evaluate-act” method.

The eighth topic (future me) is related to overcoming obstacles (treatment resistance) and developing a positive self-image, and may be configured with therapy modules to organize previously learned coping methods, create the pain patient's own recipe for coping with pain (pain coping recipe), and identify alternatives to expected difficulties.

Meanwhile, in the present invention, topics 510a to 580a may be predefined and exist in the storage unit 120. Meanwhile, it is obvious that the number and content (or kinds) of topics in the present invention are not limited to the examples described above and may be defined in various ways.

The specific therapy program may be configured to include at least one therapy module related to a specific topic matched to the specific therapy program. For example, the first therapy program 510 may be configured with a plurality of therapy modules 511 to 515 related to the first topic (e.g., “motivation enhancement”, 510).

Here, the term “therapy module” may mean contents related to a detailed category (or subtopic) for cognitive behavioral therapy of the pain patient for a specific topic. For example, the plurality of therapy modules 511 to 515 related to the first topic (e.g., “motivation enhancement”, 510a) may include “what pain means to me therapy module 511”, “pain schema therapy module 512”, “pain questionnaire therapy module 513”, “set goals therapy module 514”, and “practice recording pain therapy module 515” (see FIG. 5A). In the present invention, a therapy module included in a therapy program related to a specific topic may be referred to as a “therapy module corresponding to specific topic”. Further, in the present invention, the term “therapy module” may be used interchangeably with the term “chapter”.

Meanwhile, as illustrated in FIG. 4, there may be different therapy protocols 410a, 420a, 430a, and 440a matched to each of the plurality of user groups 410, 420, 430, and 440.

The plurality of therapy protocols 410a, 420a, 430a, and 440a matched to each of the plurality of user groups 410, 420, 430, and 440 may be configured with at least one different therapy program or therapy module, depending on the characteristics of the pain duration 450 and degree of cognitive distortion 460 of the user group 410, 420, 430, and 440.

For example, the first therapy protocol 410a matched to the first user group 410 and the third therapy protocol 430a matched to the third user group 430 may be configured to include all therapy modules (full modules) stored on the server. In this case, all therapy modules include all of the plurality of therapy modules matched to each of the first to eighth topics, and cognitive behavioral therapy may be performed using all of the therapy modules included in each of the first to eighth topics for a pain patient belonging to the first user group.

In another example, a therapy protocol matched to the second user group 420 and the fourth user group 440 may be configured to include some of the therapy modules, but not all of the therapy modules (full modules) stored on the server. The therapy protocol matched to the second user group 420 and the fourth user group 440 may include only some of the therapy modules of the plurality of therapy modules matched to each of the first to eighth topics, and cognitive behavioral therapy may be performed for the pain patient belonging to the second user group 420 and the fourth user group 440 using some of the therapy modules among the therapy modules constituting the first to eighth topics.

In one example, the second therapy protocol 420a matched to the second user group 420 may be configured to focus on modules related to coping methods, breathing techniques, and disease education. In another example, the fourth therapy protocol 440a matched to the fourth user group 440 may be configured to focus on modules related to acceptance commitment.

Meanwhile, as illustrated in FIGS. 5A and 5B, each of the plurality of therapy programs 510 to 580 may include worksheet modules 515, 522, 531, 541, 551, 561, 571, and 581 for reviewing at least one of a degree of pain, pain duration, a mental health state, or a physical health state of the patient.

In this case, user response information may be input into each of the worksheet modules for reviewing at least one of the degree of pain, pain duration, mental health state, and physical health state of the pain patient in relation to a specific topic of the therapy program in which the worksheet module is included. Further, various user response information may be input into each of the worksheet modules for reviewing at least one of the degree of pain, pain duration, mental health state, and physical health state of the pain patient, as well as contents related to a specific topic of the therapy program.

For example, the user response information may be input into the worksheet module 515 included in the first therapy program 510 to review at least one of the degree of pain, pain duration, mental health state, and physical health state of the pain patient in relation to the first topic (“motivation enhancement”, 510a).

In another example, the user response information may be input into the worksheet module 522 included in the second therapy program 520 to review at least one of the degree of pain, pain duration, mental health state, and physical health state of the pain patient in relation to the second topic (“emotion identification”, 520a).

The worksheet module may be provided at various occasions in each therapy program. For example, the worksheet module may be positioned at the very last or in the middle of each therapy module. The position where the worksheet module is included in each therapy program may vary depending on the state of the pain patient.

Meanwhile, the worksheet module may be provided as the very last module in each therapy program. The worksheet module may be disposed as the very last module among the therapy modules constituting each therapy program, so that the pain patient proceeds to the worksheet module after completing the therapy modules of the corresponding therapy program. This may include the purpose of receiving a therapy effectiveness of the pain patient, objectification, or feedback from the pain patient on the therapy program.

Further, the worksheet modules included in each of the therapy programs may be configured to allow the pain patient to score (select a score, input a score, etc.) the severity of pain, mood when experiencing pain, degree of negative emotions for pain, degree of stress for pain, and the like. The control unit 130 may judge, using the score received from the worksheet module, whether the therapy program including the worksheet module was helpful and effective for the pain patient.

For example, when there is a therapy program with a low pain score, the control unit 130 may judge that the corresponding therapy program was helpful to the user. In this case, the corresponding therapy program may be reflected in an update of the therapy protocol described below.

Meanwhile, in the present invention, a process of sequentially providing a plurality of specific therapy programs included in the initial therapy protocol to the user terminal 10 according to a therapy week set for each of the plurality of specific therapy programs may be performed (S260, see FIG. 2).

Here, the term “therapy week” may be understood as a sequence (or period) in which the therapy program is provided (or activated) through the user terminal 10 for the patient to perform cognitive behavioral therapy according to the therapy program. In the present invention, the “total number of times” of therapy weeks and the “therapy period for each week” corresponding to each therapy week may be preset and exist. Further, in the present invention, it may be understood that the “overall therapy period” is predefined. The overall therapy period may be determined as the product of the preset total number of times and the preset therapy period for each week. For example, when the total number of times is “eight” and the therapy period for each week is one week (seven days), the preset overall therapy period may be “eight weeks”.

Hereinafter, for convenience of description, an example in which a different therapy week arrives for each one-week therapy period “eight times” will be described. That is, in the present invention, a first therapy week may arrive in a first therapy session, and a second therapy week may arrive in a second therapy session. Accordingly, in the present invention, the term “therapy week” may be used interchangeably with “therapy session”, “therapy round”, “therapy period”, and “therapy sequence”.

The control unit 130 may set at least one therapy week for each of the plurality of therapy programs included in the initial therapy protocol. Further, the control unit 130 may, in a specific therapy week, provide a therapy program for which the specific therapy week is set.

As described above, in the present invention, a total number of times of a therapy week (e.g., “8”) may be predefined and exist.

The control unit 130 may set a different therapy week for each of the plurality of therapy programs when the plurality of therapy programs included in the initial therapy protocol correspond to the predefined number of times of the therapy week. For example, when a first therapy program to an eighth therapy program is selected in the initial therapy protocol, the control unit 130 may set one of the first therapy week to the eighth therapy week for each of the first therapy program to the eighth therapy program.

Meanwhile, the first topic to the eighth topic as described above may correspond to the first therapy program to the eighth therapy program, respectively. That is, i) the first therapy program may be for training or therapy related to the first topic (motivation enhancement), ii) the second therapy program may be for training or therapy related to the second topic (emotion identification), iii) the third therapy program may be for training or therapy related to the third topic (behavioral strategy), iv) the fourth therapy program may be for training or therapy related to the fourth topic (attention shift), v) the fifth therapy program may be for training or therapy related to the fifth topic (thought shift), vi) the sixth therapy program may be for training or therapy related to the sixth topic (thought record), vii) the seventh therapy program may be for training or therapy related to the seventh topic (management strategy), and viii) the eighth therapy program may be for training or therapy related to the eighth topic (future me).

Meanwhile, the control unit 130 may set a plurality of therapy weeks for at least some of the plurality of therapy programs when the plurality of therapy programs included in the initial therapy protocol falls short of the predefined number of times of the therapy weeks. The control unit 130 may repeatedly allocate the same topic to different weeks, such that the therapy is performed according to the predefined therapy week. For example, when the predefined therapy week is 8 weeks and there are seven therapy programs selected in the initial therapy protocol, the control unit may repeatedly allocate one of the seven therapy programs to a specific week. For example, the control unit 130 may repeatedly set the same topic (e.g., thought record, 560a) in the sixth week and the seventh week.

Meanwhile, the control unit 130 may, based on the initial therapy protocol, sequentially provide the plurality of specific therapy programs to the user terminal 10 according to the therapy week set for each of the plurality of specific therapy programs.

The control unit 130 may, based on the initial therapy protocol, activate a state of the specific therapy program such that, during a therapy period corresponding to the specific therapy week, the specific therapy program for which the specific therapy week is set may be provided on the user terminal 10. Hereinafter, to avoid terminological confusion, a state of a specific therapy program will be described to be referred to as a “mode” of a specific therapy program.

In the present invention, a “therapy program activation mode” may be understood as a mode in which at least some of the plurality of therapy modules included in the therapy program are available for being viewed (or used). In contrast, a “therapy program deactivation mode” may be understood as a mode in which all of the plurality of therapy modules included in the therapy program are not available for being viewed (or used).

For example, as illustrated in FIG. 5A, the control unit 130 may, based on the arrival of the first therapy week (first week), activate a mode of the first therapy program 510 in which the first therapy week is set. Further, the control unit 130 may, based on the arrival of the second therapy week (second week), activate a mode of the second therapy program 520 in which the second therapy week is set.

Accordingly, the patient may be systematically provided with cognitive behavioral therapy using the plurality of therapy programs sequentially according to the therapy week set in the initial therapy protocol.

Meanwhile, the control unit 130 may sequentially provide specific therapy modules constituting the plurality of specific therapy programs to the user terminal 10, according to the therapy week set for each of the plurality of specific therapy programs.

The control unit 130 may sequentially provide the plurality of therapy modules 511 to 515 constituting the first therapy program 510 to the user terminal 10 in the first therapy week (see FIG. 5A).

Here, “therapy module sequence” may be understood as a sequence in which the plurality of therapy modules included in a specific therapy program are provided.

Further, the “therapy module activation mode” may be understood as a mode in which the therapy module is available for being viewed (or used). In contrast, the “therapy module deactivation mode” may be understood as a mode in which the therapy module is not available for being viewed (or used).

The control unit 130 may, depending on the sequence of therapy modules, change (or switch) a mode of the therapy module corresponding to the next turn in the sequence from a deactivation mode to an activation mode based on the completion of the cognitive behavioral therapy according to the therapy module corresponding to a specific sequence.

For example, as illustrated in FIG. 5A, assume that the first therapy program 510 is configured with the first therapy module to the fifth therapy module 511 to 515. The control unit 130 may switch a mode of the second therapy module 512 from a deactivation mode to an activation mode based on the completion of the cognitive behavioral therapy of the first therapy module 511.

Meanwhile, the control unit 130 may switch a mode of the therapy module with the earliest sequence among the plurality of therapy modules constituting a specific therapy program from a deactivation mode to an activation mode based on a mode of the specific therapy program being activated.

For example, the control unit 130 may switch a mode of the first therapy module 511 corresponding to the first priority (first sequence) of the plurality of therapy modules 511 to 515 included in the first therapy program 510 from a deactivation mode to an activation mode based on the first therapy program 510 being activated.

In this case, the first priority therapy module in the second week therapy program to the eighth week therapy program 520 to 580, excluding the first week therapy program 510, may be the first therapy module in which the last worksheet module review module including the user response information for the worksheet module provided in the previous therapy week corresponds to the first priority in sequence.

The control unit 130 may, in order to make the pain patient aware of the past state of the pain patient, prioritize providing the user response information for the worksheet module provided in the therapy week prior to the current therapy week before providing the therapy program corresponding to the current therapy week among the plurality of specific therapy programs.

For example, the control unit 130 may prioritize providing the user response information for the worksheet module provided in the first therapy program to the user terminal 10 before providing the second therapy program. Further, the user response information for the worksheet module provided in the previous therapy week may be provided at various occasions.

Meanwhile, in the present invention, “specific therapy program is activated” may be understood as switching the mode of the last worksheet module corresponding to the first priority (e.g., first sequence) among the plurality of therapy modules constituting the specific therapy program from a deactivation mode to an activation mode.

Meanwhile, the control unit 130 may collect, from the user terminal 10, therapy response data from specific therapy modules.

The control unit 130 may, based on a specific therapy module being selected that is activated through the user terminal 10, provide a page corresponding to the specific therapy module to the user terminal 10.

The page corresponding to the specific therapy module may include cognitive behavioral therapy content related to a specific topic. For example, a page corresponding to the therapy module 511 included in the first therapy program 510 may include cognitive behavioral therapy content (e.g., “What pain means to me?”) related to the first topic (e.g., “motivation enhancement”, 510a). Hereinafter, for convenience of description, a page corresponding to a specific therapy module will be referred to as a “specific topic related page”.

The control unit 130 may, through a specific topic related page provided to the user terminal 10, collect therapy response data according to the cognitive behavioral performance of the pain patient related to a specific therapy module.

Then, the control unit 130 may match the therapy response data collected through the page corresponding to the specific therapy module with the specific topic and store the therapy response data in the storage unit 120. In this case, the specific topic matched with the therapy response data may mean a topic related to the specific therapy module.

For example, assume that a page corresponding to a specific therapy module (e.g., a pain recording practice page) is provided on the user terminal 10 based on a specific module (one of 511 to 515) included in the first program 510 being selected. The control unit 130 may match therapy response data input through the page above with the first topic (e.g., “motivation enhancement”, 510a) and store the therapy response data in the storage unit 120 (see FIG. 5A).

In another example, assume that a page corresponding to a specific module is provided on the user terminal 10 based on a specific module 521 or 522 included in the second program 510 being selected. The control unit 130 may match therapy response data input through the page above with the second topic (e.g., “emotion identification”, 520a) and store the therapy response data in the storage unit 120 (see FIG. 5A).

Meanwhile, the control unit 130 may configure a page such that the pain patient inputs therapy response data required by a specific therapy module on the page corresponding to a specific therapy module.

For example, the control unit 130 may, in order to collect therapy response data related to a perception of pain intensity of the pain patient, display question data questioning the pain intensity (e.g., “How is your pain today?”) in one area of the page, and graphic objects corresponding to each of a plurality of pain intensities (e.g., “Pain intensity 1 to pain intensity 5”) in a different area of the page. Further, the control unit 130 may collect therapy response data for the pain intensity of the patient based on a specific graphic object being selected on the page.

In another example, the control unit 130 may, in order to collect therapy response data for an emotion shift method of the pain patient, provide a text input field on one area of the page in which the pain patient may input an emotion shift method that the pain patient desires to attempt. The control unit 130 may collect therapy response data related to the emotion shift method that is input into the text input field.

Meanwhile, the control unit 130 may provide a page including various cognitive behavioral therapy contents on the user terminal 10 for the cognitive behavioral therapy of the pain patient, in addition to the examples described above. Further, the control unit 130 may collect various therapy response data required for the cognitive behavioral therapy through the page.

Meanwhile, in the present invention, based on a result of the cognitive behavioral therapy of the pain patient proceeding on the basis of the initial therapy protocol (in particular, initial therapy response data), a therapy program (or a therapy module constituting a therapy program) corresponding after a specific therapy week can be changed.

Accordingly, an “initial therapy protocol” as described in the present invention may mean a therapy protocol determined based on survey response data, and an “update therapy protocol” may be understood as a protocol in which at least one of an initial therapy program or an initial therapy module included in the initial therapy protocol is changed based on the initial therapy response data.

Meanwhile, as information used to update the initial therapy protocol, various information may be used in addition to information collected from the therapy modules. For example, in the present invention, after therapy according to the initial therapy protocol is initiated, an investigation (information collection) of at least one of the patient's i) degree of pain, ii) degree of negative emotion (depression), iii) presence of insomnia, iv) degree of cognitive distortion (catastrophizing, risk avoidance), v) degree of stress due to pain, vi) most discomfort issue in daily life (occupation, interpersonal relationships, etc.), and vii) competence in coping with pain may be performed through a questionnaire once daily during a preset initial therapy period (e.g., initial 4 weeks).

For example, in the present invention, after the therapy according to the initial therapy protocol is initiated, an investigation (information collection) of viii) a therapy module or method that the patient subjectively finds most helpful in coping with pain may be performed at weekly intervals during the preset initial therapy period. The control unit 130 may update the initial therapy protocol based on an analysis of the information investigated above.

Further, in order to collect the information above, the control unit 130 may provide the user with an information collection alarm in various ways (e.g., an application push message, etc.), and when an answer for collecting the information above is not made within a preset time, an additional alarm may be provided. In this case, the time required to collect the information may be limited, and the control unit 130 may impose a preset evaluation time and ensure that, within the corresponding evaluation time, an evaluation of at least one of the items i) to viii) above is made.

The user's information input for items i) to viii) above may be used as “therapy response data” as described in the present invention. Meanwhile, it is obvious that the frequency and interval at which the therapy response data is collected may be modified in various ways. Hereinafter, a method of updating the therapy protocol will be described in detail.

In the following, for convenience of description, an example with an overall therapy period of “8 weeks” will be described. However, this is an example for illustrative purposes only, and the therapy period may be variously set and changed by the patient and system administrator.

Meanwhile, the control unit 130 collects therapy response data related to the cognitive behavioral therapy according to the initial therapy protocol, and may, based on a preset initial therapy period (e.g., “4 weeks”) being elapsed out of the preset overall therapy period (e.g., “8 weeks”), update the initial therapy protocol using the collected initial therapy response data.

The control unit 130 may, in order to update the initial therapy protocol, analyze the state of the pain patient by at least one analysis category based on the initial therapy response data.

In this specification, the term “analysis category” refers to a category subject to analysis of therapy response data for the cognitive behavioral therapy of the pain patient, which may include, for example, at least one of emotion, pain, insomnia, cognitive distortion, stress, competence, or discomfort.

The control unit 130 may obtain (or derive, calculate, or compute), using the initial therapy response data, a state analysis result for at least one of the pain patient's i) degree of pain (related to a pain analysis category), ii) degree of negative emotion (related to an emotion analysis category), iii) degree of insomnia (related to an insomnia category), iv) degree of stress for pain (related to a stress analysis category), v) degree of competence in coping with pain (related to a competence category), vi) degree of discomfort in daily life, or vii) degree of cognitive distortion.

The control unit 130 may derive the state analysis result of the pain patient for each analysis category in various ways.

For example, the control unit 130 may, using an artificial intelligence model that has performed machine learning for analyzing the state of the pain patient, obtain the state analysis result of the pain patient for each analysis category. In this case, the state analysis result obtained using the artificial intelligence model may include information that specifies a specific analysis category (e.g., “insomnia”) in which a problematic symptom of the pain patient satisfies a preset reference among a plurality of analysis categories. Alternatively, the state analysis result obtained using the artificial intelligence model may include an analysis score for each of the plurality of analysis categories.

In another example, the control unit 130 may compare whether the analysis score for each of the plurality of analysis categories satisfies the preset reference to specify an analysis category for which the problematic symptom of the pain patient among the plurality of analysis categories satisfies the preset reference. The control unit 130 may calculate (or compute) the analysis score for each analysis category based on the therapy response data. For example, the control unit 130 may the analysis score using (or calculating) initial therapy response data corresponding to multiple choice questions. In addition, the control unit 130 may perform an artificial intelligence analysis of the initial response data made up of a text to calculate the analysis score for the initial response data made up of a text. The control unit 130 may compare each of the calculated analysis scores for each category to a preset problem score for each category, and specify an analysis category in which the analysis score exceeds the preset problem score as a category in which the pain patient has a problematic symptom.

Meanwhile, the control unit 130 may, using the state analysis result of the pain patient based on the initial therapy response data, update the initial therapy program such that at least one of the therapy program and the therapy module related to the specified category is included in the therapy program.

As illustrated in FIG. 7, the control unit 130 may, based on the therapy response data according to the first therapy program to the fourth therapy program 510 to 540 set in each of the first therapy week to the fourth therapy week, change at least some of the programs 550 and 560 of the fifth therapy program to the eighth therapy program 550 to 580 set in each of the fifth therapy week to the eighth therapy week to new programs 550′ and 560′.

In this case, the control unit 130 may change remaining therapy programs allocated to a remaining therapy period (5 to 8 weeks) excluding the preset initial therapy period (e.g., 1 to 4 weeks), and at least one of therapy modules constituting the remaining therapy programs, to be associated with the specified analysis category (e.g., “insomnia”).

As illustrated in FIG. 8A, assume that an initial therapy protocol 810 exists. The initial therapy protocol 810 may be configured to include a first initial therapy program to an eighth initial therapy program 811 to 818 related to different topics, at each of the first week and the eighth week. Further, each of the initial therapy programs 811 to 818 may include a therapy module corresponding to each topic. For example, the fifth initial therapy program 815 may include a plurality of initial therapy modules 815a to 815f associated with a fifth topic, and the sixth initial therapy program 816 may include a plurality of initial therapy modules 816a to 816f associated with a sixth topic.

As illustrated in FIG. 8B, the control unit 130 may, based on the state analysis result of the pain patient for the initial therapy response data, exclude at least some (815d to 815f and 816d to 8160 of the therapy modules 815a to 815f and 816a to 816f included in the remaining therapy programs 815 to 816. The remaining therapy programs 815 to 816 of an updated therapy protocol 820 may include at least some therapy modules 815a to 815c and 816a to 816c that are not excluded. In this case, the non-excluded therapy modules 815a to 815c and 816a to 816c may be associated with the specified analysis category.

Further, the control unit 130 may, based on the state analysis result of the pain patient for the initial therapy response data, reset a specific therapy program set in the preset initial therapy period (or initial therapy week) to at least a portion of the remaining therapy period (or remaining therapy week). That is, the control unit 130 may update the initial therapy protocol by reallocating (or resetting or reassigning) the therapy program associated with a specific topic matched to the preset initial therapy period (or initial therapy week) to the remaining therapy period (or remaining therapy week).

For example, as illustrated in FIG. 8C, the control unit 130 may reset the third therapy program 813 set in the third week to the fifth week, and the fourth therapy program 814 set in the fourth week to the sixth week. An updated therapy protocol 830 may include the previously performed therapy programs 813 and 814 duplicated (or repeated) at different weeks. That is, in the present invention, the initial therapy program may be updated based on the initial therapy response data such that the previously performed therapy program is performed again.

The duplicately or repeatedly performed therapy program may be a module that is judged to be most helpful to the pain patient. This judgment may be achieved through an analysis of evaluation information on the therapy program received from the pain patient, changes in values for pain (e.g., severity of pain, mood when experiencing pain, degree of negative emotions for pain, value (score) for degree of stress for pain) received through the worksheet module, frequency of use of coping method for pain created in the specific therapy program, time of use, etc. This analysis may be performed using a variety of artificial intelligence algorithms.

In this case, the reallocated therapy programs 813 and 814 may be associated with a specific analysis category.

Further, the control unit 130 may update the initial therapy program 810 such that the therapy program configured with the therapy modules corresponding to each of the different topics is set in the remaining therapy period (remaining therapy week).

For example, as shown in FIG. 8D, an updated protocol 840 may include a therapy program 841 that includes a combination of therapy modules corresponding to a plurality of topics. The therapy program 841 may include therapy modules 813a to 813c associated with a third topic and therapy modules 814a to 814c associated with a fourth topic, and may be set in the fifth therapy week. That is, at least some programs 841 of the therapy programs set in the remaining therapy period (remaining therapy week) may include some of the therapy modules provided in the preset initial therapy period (or initial therapy week).

Further, although not illustrated, the control unit 130 may update the initial therapy program by newly adding modules different from the modules set in the initial protocol to the remaining therapy period (remaining therapy week).

For example, assume that the fifth therapy program 815 constituting the initial protocol 810 included six fifth topic therapy modules 815a to 815f (see FIG. 8A). The control unit 130 may update the initial therapy protocol 810 by adding a new therapy module of the fifth topic that is not included in the therapy modules 815a to 815f according to the previously included fifth topic to the fifth therapy program 815.

In this case, the control unit 130 may exclude at least some of the previously included fifth topic therapy modules 815a to 815f and add new therapy modules. In addition, the control unit 130 may include new therapy modules, while leaving the previously included fifth topic therapy modules 815a to 815f unchanged. That is, the control unit 130 may include entirely new therapy modules that are different from the previously included therapy modules, in which case the previously included therapy modules may be excluded, retained, or modified in various ways.

Meanwhile, the control unit 130 may, based on an update result of the initial therapy protocol, change at least some of the example sentences provided by the therapy modules constituting the remaining therapy programs to be associated with a specified category.

As described above, the control unit 130 may specify, among the plurality of analysis categories, a category in which the problematic symptom of the pain patient satisfies the preset reference.

In this case, the control unit 130 may specify categories as many as a preset number (e.g., two) of the plurality of analysis categories. For example, the control unit 130 may specify the “depression” and “analysis” categories that have the highest analysis scores among the categories of emotion, pain, insomnia, cognitive distortion, stress, competence, and discomfort.

There may be example sentence information in the storage unit 120 that corresponds to at least one category that satisfies the preset reference. The control unit 130 may match at least one of information on the category satisfying the preset reference and example sentence information included in the category satisfying the preset reference to a patient's account and store it as matching information. Further, in the matching information, in addition to the categories satisfying the preset reference, information on selected example sentences selected by the pain patient among the example sentences provided in the therapy modules already performed by the pain patient may be stored. Further, the example sentences stored as the matching information for the category satisfying the preset reference may also be configured with the selected example sentences selected by the pain patient.

The control unit 130 may control such that, based on the example sentence information existing in the storage unit 120, the therapy programs 550 to 580 matched for the remaining therapy weeks include the example sentences associated with the specified analysis category. The control unit 130 may change (replace) at least some of the preset example sentences for the therapy modules constituting the therapy program matched to the remaining therapy weeks with selected example sentences stored in the example sentence information corresponding to the user history.

For example, assume that the depression category is specified. The control unit 130 may include an example sentence of “Ms. Hyeryun Jang, do you think that ‘my depressed heart can never be good again’ in a specific therapy module constituting the fifth therapy program set in the fifth week.

In another example, assume that the insomnia category is specified. The control unit 130 may include an example sentence of “Ms. Hyeryun Zhang, do you think that if you have pain, you can never sleep deeply” in a specific therapy module constituting the sixth therapy program set in the sixth week. As such, the control unit 130 may objectify the user's perception of the corresponding example sentence by continuously exposing the example sentence selected by the pain patient in the past or the example sentence of a specific category to the pain patient.

Meanwhile, in the present invention, based on the result of the cognitive behavioral therapy of the pain patient proceeding on the basis of the initial therapy protocol (in particular, the initial therapy response data), a judgment on whether to maintain the initial therapy protocol as it is or whether to change the initial therapy protocol may be performed.

The control unit 130 may, based on the result of the cognitive behavioral therapy of the pain patient, perform a judgment on whether to maintain the preset overall therapy period according to the initial therapy protocol. Further, the control unit 130 may, based on a judgment result, perform an update to the overall therapy period. Here, the update to the overall therapy period may include extending or shortening the overall therapy period. The control unit 130 may perform a judgment on whether to reduce (or stop or shorten) the overall therapy period, whether to maintain the overall therapy period as it is, or whether to extend the therapy period to a period longer than the overall therapy period.

The control unit 130 may perform the judgment above based on various references, and may make a judgment on stopping, maintaining, or extending the therapy for the pain patient, for example, based on whether the result of the cognitive behavioral therapy for the pain patient satisfies the preset reference (e.g., the reference set in relation to stopping, maintaining, or extending the therapy, respectively).

In an example, the control unit 130 may quantify the result of the cognitive behavioral therapy for the pain patient as a score or the like, and may make a judgment on stopping, maintaining, or extending the therapy for the pain patient based on which of a plurality of intervals the score is included. For example, when a result score of the cognitive behavioral therapy for the pain patient is included in a first interval (stopping interval), the control unit 130 may stop (or reduce) the cognitive behavioral therapy for the pain patient.

For example, when a result of the cognitive behavioral therapy for the pain patient is positive, the result of the cognitive behavioral therapy may be included in the score of the first interval. In this case, the control unit 130 may reduce the overall therapy period, in which case the control unit 130 may determine the period that is reduced. The control unit 130 may update the initial therapy protocol such that only the preceding therapy is performed up to an occasion when monitoring has been performed, and no subsequent therapy is performed. In this case, the cognitive behavioral therapy for the pain patient may be stopped.

In another example, when a result score of the cognitive behavioral therapy for the pain patient is included in a second interval (maintaining interval), the control unit 130 may maintain the cognitive behavioral therapy for the pain patient. In this case, the therapy period set in the initial protocol (e.g., 8 weeks) may remain the same. When the result of the cognitive behavioral therapy for the pain patient is moderate, the result of the cognitive behavioral therapy may be included in the score of the second interval. In this case, the control unit 130 may maintain the overall therapy period. Meanwhile, even in this case, the update to the initial protocol may be made, and the update to the initial protocol may be made as described above in conjunction with FIGS. 8A to 8D, and thus the specific description will be replaced by the above description. In still another example, when a result score of the cognitive behavioral therapy for the pain patient is included in a third interval (maintaining interval), the control unit 130 may extend the therapy period of the cognitive behavioral therapy for the pain patient. In this case, the therapy period set in the initial protocol (e.g., 8 weeks) may be extended (e.g., 12 weeks, etc.). When the result of the cognitive behavioral therapy for the pain patient is negative, the result of the cognitive behavioral therapy may be included in the score of the third interval. In this case, the control unit 130 may further extend the overall therapy period. The control unit 130 may determine the extended period. The control unit 130 may determine the extent to which the period is extended based on the state of the pain patient. As the state of the patient is worse, the extended period may be longer. The control unit 130 may, when the therapy period is extended, perform a determination on the therapy program to be performed during the extended therapy period, and update the initial therapy program such that the determined therapy program is further allocated to the extended therapy period. Of course, the update to the initial therapy protocol may further include an update to the already allocated therapy program, as well as a decision on programs for the extended therapy period.

Meanwhile, the control unit 130 may use feedback on the therapy program performed by the pain patient to determine which therapy program to be further allocated for the extended treatment period. Further, the control unit 130 may use the state information on the pain patient for each analysis category analyzed based on the initial therapy response data for the previously performed therapy program to allocate the therapy program for the extended treatment period. The control unit 130 may, based on the state information on the pain patient for each analysis category of at least one of emotion, pain, insomnia, cognitive distortion, stress, competence, and discomfort, determine the category in which more therapy is to be performed on the pain patient, and ensure that the therapy program for the corresponding category is further allocated to the extended treatment period. Meanwhile, a method of determining the therapy program to be allocated for the extended therapy period may vary widely. For example, the control unit 130 may receive a patient's intent for an extension of the therapy program of the patient. An occasion for receiving the patient's intent may vary, for example, the intent on whether the patient wishes to extend the therapy period may be received from the patient at an occasion when the preset therapy period is completed or at an occasion when the preset therapy program is initiated. The control unit 130 may provide an interface for receiving information from the patient, with a pop-up page or in various other ways. The control unit 130 may extend the therapy program when receiving the intent to extend the therapy period from the patient. In this case, the extended period may also be selected by the patient. Further, at least one of a topic or therapy module of the extended therapy program may be selected by the user. Therefore, the patient can receive more effective therapy by selecting a therapy program or therapy module that is beneficial to himself/herself. As described above, a method and system for providing cognitive behavioral therapy for a pain patient according to the present invention can receive, through a user terminal, a plurality of survey response data, and, based on the survey response data, detect state information of the pain patient related to pain duration and a degree of cognitive distortion of the pain patient. Further, based on the state information of the pain patient, a therapy protocol for personalized cognitive behavioral therapy can be provided to the pain patient. As a result, the method and system of cognitive behavioral therapy according to the present invention can provide a personalized cognitive behavioral therapy program for the pain patient in consideration of the pain duration and degree of cognitive distortion of the pain patient even if the pain patient has the same disease, rather than providing a uniform cognitive behavioral therapy based on the disease of the pain patient. Further, the pain patient can be provided with the personalized cognitive behavioral therapy that fits his/her state.

Further, the method and system for providing cognitive behavioral therapy for a pain patient according to the present invention may sequentially provide a plurality of specific therapy programs according to a therapy week. This allows the pain patient to be provided with cognitive behavioral therapy in a systematic way so that the pain patient can complete the cognitive behavioral therapy without dropping out.

Further, the method and system for providing cognitive behavioral therapy for a pain patient according to the present invention can update the therapy program to help treat the pain patient based on therapy data collected in the process of performing the cognitive behavioral therapy, so that the updated cognitive behavioral therapy can be provided in consideration of the pain patient's remission, rather than the initial therapy program being provided continuously.

Meanwhile, the present invention described above may be executed by one or more processes on a computer and implemented as a program that can be stored on a computer-readable medium (or recording medium).

Further, the present invention described above may be implemented as computer-readable code or instructions on a medium in which a program is recorded. That is, the present invention may be provided in the form of a program.

Meanwhile, the computer-readable medium includes all kinds of storage devices for storing data readable by a computer system. Examples of computer-readable media include hard disk drives (HDDs), solid state disks (SSDs), silicon disk drives (SDDs), ROMs, RAMs, CD-ROMs, magnetic tapes, floppy discs, and optical data storage devices.

Further, the computer-readable medium may be a server or cloud storage that includes storage and that the electronic device is accessible through communication. In this case, the computer may download the program according to the present invention from the server or cloud storage, through wired or wireless communication.

Further, in the present invention, the computer described above is an electronic device equipped with a processor, that is, a central processing unit (CPU), and is not particularly limited to any type.

Meanwhile, it should be appreciated that the detailed description is interpreted as being illustrative in every sense, not restrictive. The scope of the present invention should be determined based on the reasonable interpretation of the appended claims, and all of the modifications within the equivalent scope of the present invention belong to the scope of the present invention.

What is claimed is:

1. A method of providing cognitive behavioral therapy for a pain patient, the method comprising:
providing a plurality of survey data for diagnosing a state of a pain patient on a user terminal;
receiving, through the user terminal, survey response data for the plurality of survey data;
specifying, based on the survey response data, a user group to which the pain patient belongs among a plurality of user groups categorized according to the state of the pain patient;
determining an initial therapy protocol corresponding to the user group among a plurality of therapy protocols;
providing sequentially, to the user terminal, a plurality of initial therapy programs included in the initial therapy protocol, according to a therapy week set for each of the plurality of initial therapy programs, during an initial therapy week of a preset period among overall therapy weeks;
collecting, from the user terminal, initial therapy response data required by the plurality of initial therapy programs provided in accordance with the initial therapy week;
analyzing a state of the pain patient using the initial therapy response data;
shortening, using the analysis result, an overall therapy period for which the plurality of initial therapy programs are scheduled to be provided to the user terminal, to a period shorter than the overall therapy period, or lengthening the overall therapy period to a period longer than the overall therapy period;
updating, using the analysis result, remaining initial therapy programs corresponding to remaining therapy weeks of the plurality of initial therapy programs, excluding the initial therapy week; and
providing, to the user terminal, therapy programs in which the remaining initial therapy programs have been updated, for each of the remaining therapy weeks, during the remaining therapy weeks,
wherein the initial therapy protocol is configured with the plurality of initial therapy programs, each of the plurality of initial therapy programs being matched to a different topic for cognitive behavioral therapy of the pain patient,
wherein each of the plurality of initial therapy programs includes a plurality of initial therapy modules associated with a topic matched to each of the plurality of initial therapy programs,
wherein, in the updated therapy program, at least some of the plurality of initial therapy modules included in the remaining initial therapy programs are excluded, a therapy program associated with a specific topic among the initial therapy programs that have been provided in the initial therapy week is repeatedly allocated to the remaining therapy weeks, or a combination of initial therapy modules corresponding to each of different plurality of topics among the initial therapy programs that have been provided in the initial therapy week is allocated to the remaining therapy weeks, and
wherein the different plurality of topics for cognitive behavioral therapy for the pain patient includes at least one of a topic related to motivation enhancement, a topic related to emotion identification, a topic related to behavioral strategy, a topic related to attention shift, a topic related to thought shift, a topic related to thought record, a topic related to management strategy, or a topic related to future me.

2. The method of claim 1, wherein each of the plurality of therapy protocols are configured with at least one different therapy program or therapy module depending on characteristics of pain duration and degree of cognitive distortion of a user group matched to each of the plurality of therapy protocols.

3. The method of claim 2, wherein the plurality of user groups comprises:
a first user group having acute pain and high cognitive distortion, in relation to the characteristics of pain duration and degree of cognitive distortion;
a second user group having acute pain and low cognitive distortion, in relation to the characteristics of pain duration and degree of cognitive distortion;
a third user group having chronic pain and high cognitive distortion, in relation to the characteristics of pain duration and degree of cognitive distortion; and
a fourth user group having chronic pain and low cognitive distortion, in relation to the characteristics of pain duration and degree of cognitive distortion,
wherein a reference for distinguishing between acute pain and chronic pain is determined by whether the pain duration exceeds a reference period, and
wherein a reference for distinguishing between high cognitive distortion and low cognitive distortion is determined by whether a score collected based on a plurality of questions related to cognitive distortion exceeds a reference score.

4. The method of claim 2, wherein the initial therapy modules constituting the plurality of initial therapy programs are provided to the user terminal, sequentially, in accordance with an initial therapy week set for each of the initial therapy programs.

5. The method of claim 1, wherein the analyzing of the state of the pain patient is performed for each of a plurality of different analysis categories related to at least one of emotion, pain, insomnia, cognitive distortion, or stress, and
wherein the updating of the remaining initial therapy programs comprises:
specifying, using the analysis result, a category of the plurality of analysis categories for which a problematic symptom of the pain patient satisfies a preset reference; and
changing at least one of the remaining initial therapy programs allocated to the remaining therapy weeks or initial therapy modules constituting the remaining initial therapy programs, to be related to the specified category.

6. The method of claim 5, wherein the updating of the remaining initial therapy programs further comprises changing at least some of example sentences provided from the initial therapy modules constituting the remaining initial therapy programs to be related to the specified category.

7. The method of claim 1, wherein each of the initial therapy programs and the updated therapy programs comprises a worksheet module configured to inspect at least one of a degree of pain, pain duration, a mental health state, or a physical health state of the pain patient, and
wherein the worksheet module, in order to make the pain patient aware of a past state of the pain patient, prior to providing a specific therapy program corresponding to a current therapy week of the initial therapy programs and the updated therapy programs, preferentially provides user response information for a worksheet module provided in a therapy week prior to the current therapy week.

8. A system for providing cognitive behavioral therapy, the system comprising:
a communication unit configured to perform communication with a user terminal;
a storage unit configured to store a plurality of therapy programs each matched to a different topic; and
a control unit configured to provide a plurality of survey data for diagnosing a state of a pain patient on the user terminal,
wherein the control unit is configured to:
receive, through the communication unit, response data to the plurality of survey data from the user terminal;
specify, based on the response data, a user group corresponding to state information on the pain patient among a plurality of user groups categorized according to the state of the pain patient;
determine an initial therapy protocol corresponding to the user group among a plurality of therapy protocols;
provide sequentially, to the user terminal, a plurality of initial therapy programs included in the initial therapy protocol, according to a therapy week set for each of the plurality of initial therapy programs, during an initial therapy week of a preset period among overall therapy weeks;
collect, from the user terminal, initial therapy response data required by the plurality of initial therapy programs provided in accordance with the initial therapy week;
analyze a state of the pain patient using the initial therapy response data;
shorten, using the analysis result, an overall therapy period for which the plurality of initial therapy programs are scheduled to be provided to the user terminal, to a period shorter than the overall therapy period, or lengthen the overall therapy period to a period longer than the overall therapy period;
update, using the analysis result, remaining initial therapy programs corresponding to remaining therapy weeks of the plurality of initial therapy programs, excluding the initial therapy week; and
provide, to the user terminal, therapy programs in which the remaining initial therapy programs have been updated, for each of the remaining therapy weeks, during the remaining therapy weeks,
wherein the initial therapy protocol is configured with the plurality of initial therapy programs, each of the plurality of initial therapy programs being matched to a different topic for cognitive behavioral therapy of the pain patient,
wherein each of the plurality of initial therapy programs includes a plurality of initial therapy modules associated with a topic matched to each of the plurality of initial therapy programs,
wherein, in the updated therapy program, at least some of the plurality of initial therapy modules included in the remaining initial therapy programs are excluded, a therapy program associated with a specific topic among the initial therapy programs that have been provided in the initial therapy week is repeatedly allocated to the remaining therapy weeks, or a combination of initial therapy modules corresponding to each of different plurality of topics among the initial therapy programs that have been provided in the initial therapy week is allocated to the remaining therapy weeks, and
wherein the different plurality of topics for cognitive behavioral therapy for the pain patient includes at least one of a topic related to motivation enhancement, a topic related to emotion identification, a topic related to behavioral strategy, a topic related to attention shift, a topic related to thought shift, a topic related to thought record, a topic related to management strategy, or a topic related to future me.

9. A program executed by one or more processes on an electronic device and stored on a computer-readable recording medium,
wherein the program comprises instructions for performing of:
providing a plurality of survey data for diagnosing a state of a pain patient on a user terminal;
receiving, through the user terminal, survey response data for the plurality of survey data;
specifying, based on the survey response data, a user group to which the pain patient belongs among a plurality of user groups categorized according to the state of the pain patient;
determining an initial therapy protocol corresponding to the user group among a plurality of therapy protocols;
providing sequentially, to the user terminal, a plurality of initial therapy programs included in the initial therapy protocol, according to a therapy week set for each of the plurality of initial therapy programs, during an initial therapy week of a preset period among overall therapy weeks;
collecting, from the user terminal, initial therapy response data required by the plurality of initial therapy programs provided in accordance with the initial therapy week;
analyzing a state of the pain patient using the initial therapy response data;
shortening, using the analysis result, an overall therapy period for which the plurality of initial therapy programs are scheduled to be provided to the user terminal, to a period shorter than the overall therapy period, or lengthening the overall therapy period to a period longer than the overall therapy period;
updating, using the analysis result, remaining initial therapy programs corresponding to remaining therapy weeks of the plurality of initial therapy programs, excluding the initial therapy week; and
providing, to the user terminal, therapy programs in which the remaining initial therapy programs have been updated, for each of the remaining therapy weeks, during the remaining therapy weeks,
wherein the initial therapy protocol is configured with the plurality of initial therapy programs, each of the plurality of initial therapy programs being matched to a different topic for cognitive behavioral therapy of the pain patient,
wherein each of the plurality of initial therapy programs includes a plurality of initial therapy modules associated with a topic matched to each of the plurality of initial therapy programs, wherein, in the updated therapy program, at least some of the plurality of initial therapy modules included in the remaining initial therapy programs are excluded, a therapy program associated with a specific topic among the initial therapy programs that have been provided in the initial therapy week is repeatedly allocated to the remaining therapy weeks, or initial therapy modules corresponding to each of different plurality of topics among the initial therapy programs that have been provided in the initial therapy week is allocated in combination to the remaining therapy weeks, and
wherein the different plurality of topics for cognitive behavioral therapy for the pain patient includes at least one of a topic related to motivation enhancement, a topic related to emotion identification, a topic related to behavioral strategy, a topic related to attention shift, a topic related to thought shift, a topic related to thought record, a topic related to management strategy, or a topic related to future me.
